SEQUENCE LISTING (11 sequences)

SEQ ID NO: 1 - protein, 118 aa, Artificial Sequence
  Synthetic Peptide
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 2 - protein, 729 aa, Artificial Sequence
  Synthetic Peptide
CELSPINASHPVQALMESFTVLSGCASRGTTGLPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPDHPPALDNPLFPGEGSPNGGLPFPFPDIPRRGWKEGEDRIPRPKQPIVPSVQLLPDHREPEEVQGGVDIALSVKCDHEKMVVAVDKDSFQTNGYSGMELTLLDPSCKAKMNGTHFVLESPLNGCGTRHRRSTPDGVVYYNSIVVQAPSPGDSAGWPDGYEDLEAGDNGFPGDGDEGETAPLSRAGVVVFNCSLRQLRNPSGFQGQLDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVD

SEQ ID NO: 3 - protein, 353 aa, Artificial Sequence
  Synthetic Peptide
CELSPINASHPVQALMESFTVLSGCASRGTTGLPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPD

SEQ ID NO: 4 - protein, 377 aa, Artificial Sequence
  Synthetic Peptide
DHPPALDNPLFPGEGSPNGGLPFPFPDIPRRGWKEGEDRIPRPKQPIVPSVQLLPDHREPEEVQGGVDIALSVKCDHEKMVVAVDKDSFQTNGYSGMELTLLDPSCKAKMNGTHFVLESPLNGCGTRHRRSTPDGVVYYNSIVVQAPSPGDSAGWPDGYEDLEAGDNGFPGDGDEGETAPLSRAGVVVFNCSLRQLRNPSGFQGQLDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVD

SEQ ID NO: 5 - protein, 206 aa, Artificial Sequence
  Synthetic Peptide
DHPPALDNPLFPGEGSPNGGLPFPFPDIPRRGWKEGEDRIPRPKQPIVPSVQLLPDHREPEEVQGGVDIALSVKCDHEKMVVAVDKDSFQTNGYSGMELTLLDPSCKAKMNGTHFVLESPLNGCGTRHRRSTPDGVVYYNSIVVQAPSPGDSAGWPDGYEDLEAGDNGFPGDGDEGETAPLSRAGVVVFNCSLRQLRNPSGFQGQL

SEQ ID NO: 6 - protein, 171 aa, Artificial Sequence
  Synthetic Peptide
DGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVD

SEQ ID NO: 7 - protein, 545 aa, Artificial Sequence
  Synthetic Peptide
MKWVTFLLLLFISGSAFSAAACELSPINASHPVQALMESFTVLSGCASRGTTGLPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPDDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVD

SEQ ID NO: 8 - protein, 886 aa, Artificial Sequence
  Synthetic Peptide
MKWVTFLLLLFISGSAFSAAANGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAACELSPINASHPVQALMESFTVLSGCASRGTTGLPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPDHPPALDNPLFPGEGSPNGGLPFPFPDIPRRGWKEGEDRIPRPKQPIVPSVQLLPDHREPEEVQGGVDIALSVKCDHEKMVVAVDKDSFQTNGYSGMELTLLDPSCKAKMNGTHFVLESPLNGCGTRHRRSTPDGVVYYNSIVVQAPSPGDSAGWPDGYEDLEAGDNGFPGDGDEGETAPLSRAGVVVFNCSLRQLRNPSGFQGQLDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVD

SEQ ID NO: 9 - protein, 681 aa, Artificial Sequence
  Synthetic Peptide
MKWVTFLLLLFISGSAFSAAANGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAACELSPINASHPVQALMESFTVLSGCASRGTTGLPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPDDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVD

SEQ ID NO: 10 - protein, 21 aa, Artificial Sequence
  Synthetic Peptide
MKWVTFLLLLFISGSAFSAAA

SEQ ID NO: 11 - protein, 18 aa, Artificial Sequence
  Synthetic Peptide
GLGPVESSPGHGLDTAAA

(19) United States
(12) Patent Application Publication
(10) Pub. No.: US 2017/0166624 A1
(43) Pub. Date: Jun. 15, 2017

(54) TGFß TYPE II-TYPE III RECEPTOR FUSIONS

(71) Applicant: THE BOARD OF REGENTS OF THE UNIVERSITY OF TEXAS SYSTEM, Austin, TX (US)

(72) Inventors: Andrew Hinck, San Antonio, TX (US); LuZhe Sun, San Antonio, TX (US); Christian Zwieb, San Antonio, TX (US)

(21) Appl. No.: 15/325,831

(22) PCT Filed: Jul. 14, 2015
(86) PCT No.: PCT/US2015/040345

§ 371 Date: Jan. 12, 2017

Related U.S. Application Data
(60) Provisional application No. 62/024,253, filed on Jul. 14, 2014.

(51) Int. Cl. C07K 14/71

(52) U.S. Cl. CPC C07K 14/71; C07K 2319/21; A61K 38/00

(57) ABSTRACT

Certain embodiments are directed to TGF-β inhibitors EUc and REUc. EUc is generated by removing the non-binding N-terminal subdomain from the TGF-β type III receptor, and REUc is generated by fusing together the binding domains of the TGF-β type II (RII or R) and type III receptor (RIII or EU) by a flexible linker and by removing the non-binding N-terminal subdomain from the TGF-β type III receptor.

This application claims priority to U.S. Provisional Patent Application Ser. No. 62/024,253 filed Jul. 14, 2014, which is incorporated herein by reference in its entirety.

### STATEMENT REGARDING FEDERALLY FUNDED RESEARCH

This invention was made with government support under CA172886, CA079683 awarded by the National Cancer Institute and GM58670 awarded by National Institute of General Medical Sciences, respectively. The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Transforming growth factor beta (TGFβ) isoforms (β1, β2, and β3) are homodimeric polypeptides of 25 kDa. TGFβs have been shown to be potent growth inhibitors in various cell types including epithelial cells (Lyons and Moses, Eur. J. Biochem. 187, 467-473, 1990). The mechanism of the growth inhibition by TGFβ is mainly due to the regulation of cell cycle-related proteins (Derynck, Trends. Biochem. Sci. 19, 548-553, 1994; Miyazono et al., Semin. Cell Biol. 5, 389-398, 1994). Thus, aberrant regulation of cell cycle machinery such as loss of retinoblastoma gene product during tumorigenesis can lead to loss of growth inhibition by TGFβ. Furthermore, mutational inactivation of TGFβ receptors, Smad2, and Smad4 has been reported in various carcinomas (Massague et al., Cell 103, 295-309, 2000). For example, loss of RI and/or RII expression is often observed in some human gastrointestinal cancers (Markowitz and Roberts, Cytokine, Growth Factor, Rev. 7, 93-102, 1996).

While many carcinoma cells lose response to TGFβ's growth inhibition, they often overproduce active TGFβ isoforms when compared to their normal counterpart (Reiss, Microbes and Infection 1, 1327-1347, 1999). This is likely to result in the selection of cancer cells that are resistant to TGFβ's growth inhibitory activity. Indeed, an increased level of TGFβ1 is strongly associated with the progression of many types of malignancies and poor clinical outcome (Reiss, Microbes and Infection 1, 1327-1347, 1999). For example, serum TGFβ1 levels have been shown to correlate to tumor burden, metastasis, and serum prostate specific antigen (PSA) in prostate cancer patients (Adler et al., J. Urol. 161, 182-187, 1999; Shariat et al., J. Clin. Oncol. 19, 2856-2864, 2001). Consistent with these observations, marked increase of TGFβ1 and TGFβ2 expression was observed in an aggressive androgen-independent human prostate cancer cell line when compared to its less aggressive androgen-dependent parent cell line, LNCap (Patel et al., J. Urol. 164, 1420-1425, 2000).

Several mechanisms are believed to mediate TGFβ's tumor-promoting activity (Arteaga et al., Breast Cancer Res. Treat. 38, 49-56, 1996; Reiss, Microbes and Infection 1, 1327-1347, 1999). TGFβ is a potent immune suppressor (Sosroseno and Herminajeng, Br. J. Biomed. Sci. 52, 142-148, 1995). Overexpression of TGFβ1 in the rat prostate cancer cells was associated with a reduced immune response during tumor formation suggesting that TGFβ may suppress host immune response to the growing tumor (Lee et al., Prostate 39, 285-290, 1999). TGFβ has also been shown to be angiogenic in vivo (Fajardo et al., Lab. Invest. 74, 600-608, 1996; Yang and Moses, J. Cell Biol. 111, 731-741, 1990; Wang et al., Proc. Natl. Acad. Sci. U.S.A. 96, 8483-8488, 1999). Overexpression of TGFβ during cancer progression is often associated with increased angiogenesis and metastasis suggesting that TGFβ may promote metastasis by stimulating tumor blood vessel formation (Roberts and Wakefield, Proc. Natl. Acad. Sci. U.S.A. 100, 8621-8623, 2003). TGFβ also plays an important role in promoting bone metastasis of human prostate and breast cancers (Koeneman et al., Prostate 39, 246-261, 1999; Yin et al., J. Clin. Invest 103, 197-206, 1999). Both TGFβ1 and TGFβ2 are produced by bone tissue, which is the largest source of TGFβ in the body (Bonewald and Mundy, Clin. Orthop. 261-276, 1990). The latent TGFβ can be activated by proteases such as PSA and urokinase plasminogen activator, which are abundantly secreted by cancer cells (Koeneman et al., Prostate 39, 246-261, 1999). Taken together, TGFβ can act in tumor microenvironment to promote carcinoma growth, angiogenesis, and metastasis.

Because of its involvement in the progression of various diseases, TGFβ has been targeted for the development of novel therapeutic strategies. One way of antagonizing TGFβ activity is to utilize the ectodomain of TGFβ type II receptor or type III receptor (betaglycan). It has previously been shown that ectopic expression of the type III receptor ectodomain in human carcinoma cell lines can significantly inhibit tumor growth, angiogenesis, and metastasis when they are inoculated in athymic nude mice (Bandyopadhyay et al., Cancer Res. 59, 5041-5046, 1999; Bandyopadhyay et al., Oncogene 21, 3541-3551, 2002b). More recently, it has been shown that systemic administration of recombinant RIII ectodomain can inhibit the growth, angiogenesis, and metastasis of the xenografts of human breast carcinoma MDA-MB-231 cells in nude mice (Bandyopadhyay et al., Cancer Res. 62, 4690-4695, 2002a). However, the inhibition was only partial. This could be due, in part, to the fact that the cells produced active TGFβ1 and active TGFβ2 and the anti-TGFβ potency of RIII ectodomain is 10-fold lower for TGFβ1 than for TGFβ2 (Vilchis-Landeros et al., Biochem. J. 355, 215-222, 2001).

While numerous TGFβ antagonists have been prepared and tested, all have less than complete TGFβ isoform inhibiting properties. Thus, there is a need for additional TGFβ antagonists or inhibitors.

### SUMMARY

Certain embodiments are directed to heteromeric polypeptides (heteromeric fusion proteins) comprising, from amino terminus to carboxy terminus (a) an ectodomain of TGF-β type II receptor (RII or R), a TGFβ receptor type III endoglin domain (E), and a TGFβ receptor type III uromodulin-like carboxy terminal binding subdomain (U_{C}) (REU_{C} polypeptide); or (b) an amino terminal TGFβ receptor type III endoglin domain (E) coupled to a TGFβ receptor type III uromodulin-like carboxy terminal binding subdomain (U_{C}) (EU_{C} polypeptide). These fusion proteins or polypeptides, e.g., REU_{C} or EU_{C}, bind TGF-β isoforms with higher affinity than either R or EU alone. Increased affinity of REU_{C} for binding TGF-β isoforms also increase their ability to antagonize TGF-β isoforms. Fusion of R onto the N-terminus of EU and deletion of one or more amino acids of the TGFβ receptor type III uromodulin-like amino terminal non-binding subdomain (U_{N}) led to an increase in inhibitory potency, with REU_{C} being roughly 8 orders, 4 orders, and 2 orders of magnitude more potent than EU, EU_{C}, and REU respectively.

The polypeptides described herein can further comprise one or more linker amino acids between one or more of (i) the amino terminal ectodomain of TGFβ receptor type II (R) and the TGFβ receptor type III endoglin domain (E), or (ii) the TGFβ receptor type III endoglin domain (E) and the uromodulin-like carboxy terminal binding subdomain (U_{C}). In certain aspects the polypeptide comprises one or more linker amino acids between all domains and subdomains of the polypeptide.

In a further aspect one or more amino acids from the TGFβ receptor type III uromodulin-like non-binding amino terminal subdomain are deleted. In certain aspects the ectodomain of TGFβ receptor type II comprises an amino acid sequence that is 90% identical to SEQ ID NO:1. The TGFβ receptor type III endoglin domain can have an amino acid sequence that is 90% identical to SEQ ID NO:3. The TGFβ receptor type III uromodulin-like domain (U) can have an amino acid sequence that is 90% identical to SEQ ID NO:4. The TGFβ receptor type III uromodulin-like amino terminal non-binding subdomain (U_{N}) can have an amino acid sequence that is 90% identical to SEQ ID NO:5. The TGFβ receptor type III uromodulin-like carboxy terminal binding subdomain (U_{C}) can have an amino acid sequence that is 90% identical to SEQ ID NO:6.

Certain embodiments are directed to polypeptides comprising an ectodomain of TGF-β type III in which one or more amino acids of the ectodomain of TGF-β type III uromodulin-like amino non-binding subdomain (U_{N}) is deleted. This protein known as EU_{C} has an increased ability to antagonize TGF-β isoforms. Deletion of one or more amino acids of the TGF-β type III receptor uromodulin-like amino terminal non-binding subdomain (U_{N}) led to an apparent increase in its inhibitory potency, with EU_{C} being roughly 0.5 to 1 orders of magnitude more potent than EU.

Polypeptides described herein can further comprise an amino terminal signal sequence. In further aspects a polypeptide can further comprise an amino terminal or carboxy terminal tag. In certain aspects a polypeptide comprises a carboxy terminal hexa-histidine tag.

An example of a TGFβ type II receptor ectodomain is provided as SEQ ID NO:1. The TGFβ type II receptor ectodomain portion of a polypeptide described herein can comprise an amino acid segment that is 85, 90, 95, 98, or 100% identical, including all values and ranges there between, to amino acids 35, 40, 41, 42, 43, 44, 45, 46, 47, 48, 49, 50, 55, 60, 65, 70, or 75 to 145, 150, 155, 160, 161, 162, 163, 164, 165, 166, 167, 168, 169, or 170 of SEQ ID NO:1, including all values and ranges there between. The polypeptide segment's ability to bind TGFβ can be determined by using standard ligand binding assays known to those of skill in the art. Thus, certain aspects include variants of the TGFβ type II receptor ectodomain that maintain sufficient binding affinity for TGFβ molecules, e.g., human TGFβs.

An example of a TGFβ type III receptor ectodomain is provided as SEQ ID NO:2. Amino acids 24-383 of SEQ ID NO:2 or SEQ ID NO:3 define the endoglin-like domain (E), amino acids 430-759 of SEQ ID NO:2 or SEQ ID NO:4 define the uromodulin-like domain (U). The polypeptide segment's ability to bind TGFβ can be determined by using standard ligand binding assays known to those of skill in the art. Thus, certain aspects include variants of the TGFβ type III receptor ectodomain and its sub domains, independently, that maintain sufficient binding affinity for TGFβ molecules, e.g., human TGFβs.

In certain aspects, the fusion protein can further comprise a linker between the structured binding domains. In a further aspect, the linkers can comprise 1, 2, 3, 4, 5, 6, 7, 8, 9, 10, 11, 12, 13, 14, 15, 16, 17, 18, 19, 20 or more amino acids. In certain aspects, the amino acids of the linker are additional TGFβ receptor type II or type III amino acid sequences. In other aspects, the linkers are not TGFβ receptor type II or type III amino acid sequences, i.e., heterologous linkers.

In certain aspects, the TGFβ type II receptor ectodomain comprises an amino acid sequence that is 85, 90, 95, 98, or 100% identical to SEQ ID NO:1, including all values and ranges there between.

In yet a further aspect, the TGFβ type III receptor ectodomain comprises an amino acid sequence that is 85, 90, 95, 98, or 100% identical to all or part of SEQ ID NO:2, including all values and ranges there between.

In certain aspects, the fusion protein or heteromeric polypeptide has an amino acid sequence that is 85, 90, 95, 98, or 100% identical to SEQ ID NO:7, SEQ ID NO:8, or SEQ ID NO:9, including all values and ranges there between.

In a further aspect, the fusion protein can further comprise an amino terminal signal sequence. In certain aspects, the fusion protein can further comprise an amino terminal or carboxy terminal tag. In certain aspects the tag is hexa-histidine.

A peptide tag as used herein refers to a peptide sequence that is attached (for instance through genetic engineering) to another peptide or a protein, to provide a function to the resultant fusion. Peptide tags are usually relatively short in comparison to a protein to which they are fused; by way of example, peptide tags are four or more amino acids in length, such as, 5, 6, 7, 8, 9, 10, 15, 20, or 25 or more amino acids. Usually a peptide tag will be no more than about 100 amino acids in length, and may be no more than about 75, no more than about 50, no more than about 40, or no more than about 30.

Peptide tags confer one or more different functions to a fusion protein (thereby “functionalizing” that protein), and such functions can include (but are not limited to) antibody binding (an epitope tag), purification, translocation, targeting, and differentiation (e.g., from a native protein). In addition, a recognition site for a protease, for which a binding antibody is known, can be used as a specifically cleavable epitope tag. The use of such a cleavable tag can provide selective cleavage and activation of a protein. Alternatively the system developed by in the Dowdy laboratory (Vocero-Akbani et al, Nat Med. 5:29-33, 1999) could be use to provide specificity of such cleavage and activation.

Detection of the tagged molecule can be achieved using a number of different techniques. These include: immunohistochemistry, immunoprecipitation, flow cytometry, immunofluorescence microscopy, ELISA, immunoblotting (“western”), and affinity chromatography.

Epitope tags add a known epitope (antibody binding site) on the subject protein, to provide binding of a known and often high-affinity antibody, and thereby allowing one to specifically identify and track the tagged protein that has been added to a living organism or to cultured cells. Examples of epitope tags include the myc, T7, GST, GFP, HA (hemagglutinin) and FLAG tags. The first four examples are epitopes derived from existing molecules. In contrast, FLAG is a synthetic epitope tag designed for high antigenicity (see, e.g., U.S. Pat. Nos. 4,703,004 and 4,851,341).

Purification tags are used to permit easy purification of the tagged protein, such as by affinity chromatography. A well-known purification tag is the hexa-histidine (6×His) tag, literally a sequence of six histidine residues. The 6×His protein purification system is available commercially from QIAGEN (Valencia, Calif.), under the name of QIAexpress®.

Certain embodiments are directed to the therapeutic use of the fusions proteins or heteromeric polypeptides described herein. Certain aspects are directed to a method of treating a TGFβ related condition comprising administering an effective amount of a fusion protein described herein. The fusion protein can be administered to a subject, such as a mammal. The mammal being treated may have or may be at risk for one or more conditions associated with an excess of TGF-β for which a reduction in TGF-β levels may be desirable. Such conditions include, but are not limited to, fibrotic diseases (such as glomerulonephritis, neural scarring, dermal scarring, pulmonary fibrosis (e.g., idiopathic pulmonary fibrosis), lung fibrosis, radiation-induced fibrosis, hepatic fibrosis, myelofibrosis), peritoneal adhesions, hyperproliferative diseases (e.g., cancer), burns, immune-mediated diseases, inflammatory diseases (including rheumatoid arthritis), transplant rejection, Dupuytren's contracture, and gastric ulcers. In certain aspects the fusion protein is administer intravascularly.

Other terms related to the description provided herein include:

The term “receptor” denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction.

By “multimeric” or “heteromultimeric” is meant comprising two or more different subunits. A “heterodimeric” polypeptide contains two different subunits, wherein a “heterotrimeric” molecule comprises three subunits.

By “soluble” multimeric receptor is meant herein a multimeric receptor, each of whose subunits comprises part or all of an extracellular domain of a receptor, but lacks part or all of any transmembrane domain, and lacks all of any intracellular domain. In general, a soluble receptor of the invention is soluble in an aqueous solution.

A “fusion” protein or heteromeric polypeptide is a protein comprising two polypeptide segments linked by a peptide bond, produced, e.g., by recombinant processes.

As used herein, a “variant” polypeptide of a parent or wild-type polypeptide contains one or more amino acid substitutions, deletions and/or additions as compared to the parent or wild-type. Typically, such variants have a sequence identity to the parent or wild-type sequence of at least about 90%, at least about 95%, at least about 96%, at least about 97%, 98%, or at least about 99%, and have preserved or improved properties as compared to the parent or wild-type polypeptide. Some changes may not significantly affect the folding or activity of the protein or polypeptide; conservative amino acid substitutions, as are well known in the art, changing one amino acid to one having a side-chain with similar physicochemical properties (basic amino acid: arginine, lysine, and histidine; acidic amino acids: glutamic acid, and aspartic acid; polar amino acids: glutamine and asparagine; hydrophobic amino acids: leucine, isoleucine, valine; aromatic amino acids: phenylalanine, tryptophan, tyrosine; small amino acids: glycine, alanine, serine, threonine, methionine), small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A (Nilsson et al., EMBO 1985, 14:1075; Nilsson et al., Methods Enzymol. 1991, 198:3), glutathione S-transferase (Smith and Johnson, Gene 1988; 67:31 et seq.), or other antigenic:epitope or binding domain. See, in general Ford et al., Protein Expression and Purification 1991, 2:95-107. DNAs encoding affinity tags are available from commercial suppliers.

Sequence differences or “identity,” in the context of amino acid sequences, can be determined by any suitable technique, such as (and as one suitable selection in the context of this invention) by employing a Needleman-Wunsch alignment analysis (see Needleman and Wunsch, J. Mol. Biol. (1970) 48:443453), such as is provided via analysis with ALIGN 2.0 using the BLOSUM50 scoring matrix with an initial gap penalty of −12 and an extension penalty of −2 (see Myers and Miller, CABIOS (1989) 4:11-17 for discussion of the global alignment techniques incorporated in the ALIGN program). A copy of the ALIGN 2.0 program is available, e.g., through the San Diego Supercomputer (SDSC) Biology Workbench. Because Needleman-Wunsch alignment provides an overall or global identity measurement between two sequences, it should be recognized that target sequences which may be portions or subsequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147:195-197), which can be obtained through available programs (other local alignment methods that may be suitable for analyzing identity include programs that apply heuristic local alignment algorithms such as FastA and BLAST programs).

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as polypeptides or peptides may be conjugated or linked covalently or noncovalently to other moieties such as polypeptides, proteins, peptides, supports, fluorescence moieties, or labels. The term “conjugate” is broadly used to define the operative association of one moiety with another agent and is not intended to refer solely to any type of operative association, and is particularly not limited to chemical “conjugation.” Recombinant fusion proteins are particularly contemplated.

The term “providing” is used according to its ordinary meaning to indicate “to supply or furnish for use.” In some embodiments, the protein is provided directly by administering the protein, while in other embodiments, the protein is effectively provided by administering a nucleic acid that encodes the protein. In certain aspects the invention contemplates compositions comprising various combinations of nucleic acid, antigens, peptides, and/or epitopes.

An effective amount means an amount of active ingredients necessary to treat, ameliorate, or mitigate a disease or a condition related to a disease. In more specific aspects, an effective amount prevents, alleviates, or ameliorates symptoms of disease, or prolongs the survival of the subject being treated, or improves the quality of life of an individual. Determination of the effective amount is well within the capability of those skilled in the art, especially in light of the detailed disclosure provided herein. For any preparation used in the methods of the invention, an effective amount or dose can be estimated initially from in vitro studies, cell culture, and/or animal model assays. For example, a dose can be formulated in animal models to achieve a desired response or circulating fusion protein concentration. Such information can be used to more accurately determine useful doses in humans.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Structure of TGF-β type III receptor, RIII, also known as EU based on its two component TGF-β binding domains, the endoglin-like or E-domain (E) and the uromodulin-like or U-domain (U). RIII's U-domain can be further subdivided into a non-binding N-terminal subdomain, designated U_{N}, and a binding C-terminal subdomain, designated U_{C}. Shown below RIII (EU) are three new TGF-β inhibitors, REU, EU_{C}, and REUc, all of which are derivatives of RIII (EU).

FIG. 2. RII (R) and RIII (EU) form a 1:1:1 complex with TGF-β homodimers. (a) SPR sensorgrams in which increasing concentrations of RII and RIII were injected over a SPR sensor surface with immobilized TGF-β2TM. Mass normalized sensorgrams are shown in panels on the left. Plots of the mass normalized equilibrium response (R_{eq}) as a function of receptor concentration ([Receptor]), along with fits to R_{eq}=(Rₘₐₓ×[Receptor])/(K_{d}+[Receptor]), are shown on the right. (b) SPR sensorgrams in which increasing concentrations of RII were injected over immobilized TGF-β2TM in the absence (left) or presence (middle) of a saturating concentration (80 nM) of RIM Plots of the mass normalized equilibrium response (R_{eq}) as a function of receptor concentration ([Receptor]), along with fits to R_{eq}=(Rₘₐₓ×[Receptor])/(K_{d}+[Receptor]), are shown on the right.

FIG. 3. Proposed structure of the 1:1:1 RII:RIII:TGF-β2TM complex and evidence that this complex forms in solution and is stable. (a) Proposed structure of the 1:1:1 RII:RIII:TGF-β2TM complex. (b) Isolation of the RII:RIII:TGF-β2TM complex using size exclusion chromatography. Peak a corresponds to the RII:RIII:TGF-β2TM complex, peak b to the RII:TGF-β2TM complex, and peak c to RII alone (as shown on the SDS-gel inset). (c) Native gel showing that the isolated RII:RIII:TGF-β2TM complex (peak a) is identical to the complex formed by adding an excess of RII and TGF-β2TM to RIII. (d) Demonstration that RII:RIII:TGF-β2TM complexes are present in a 1:1:1 molar ratio based on SDS-PAGE analysis of the complex isolated by size exclusion chromatography relative to standard amounts of the individual components (left) and quantitating the relative amounts of RII:RIII:TGF-β2TM in the complex using densitometry (right).

FIG. 4. The N-terminal subdomain of the RIII U-domain (U_{N}) is dispensable for binding TGF-β based on near identical SPR sensorgrams obtained upon injection of increasing concentrations of the full-length RIII U-domain (U, or U_{N}-U_{C}) or only the C-terminal portion of the RIII U-domain (U_{C}) over immobilized TGF-β2TM.

FIG. 5. SDS-PAGE analysis (2 μg each) of the isolated inhibitors used for binding and inhibition studies. Samples of EU and REU were produced in stably transfected CHO cells, while samples of EU_{C} and REU_{C} were produced in transiently-transfected HEK-293F ‘Freestyle’ cells (Invitrogen, Carlsbad, Calif.).

FIG. 6. SPR competition binding data in which increasing concentrations of RII (R), RIII (EU), and RII-RIII (REU) were pre-incubated with 0.8 nM TGF-β3 for 16 h and then injected over a high-density (20000 RU) SPR surface with the TGF-β monoclonal antibody 1D11. Data is presented in terms of the initial slope (which is directly proportional to the concentration of the free TGF-β3 concentration) as a function of the competitor (R, EU, or REU) concentration. Two independent measurements were performed for each of the receptor constructs studied (designated by −a and −b in the legend).

FIG. 7. Inhibition of TGF-β1 induced phosphorylation of Smad2 and Smad3 in cultured MD-MBA-231 breast epithelial cells by EU, REU, EU_{C}, REU_{C}, and the neutralizing antibody 1D11. Cultured cells in serum free medium were treated with inhibitor for 5 minutes at the concentration indicated, followed by addition of TGF-β1 to a final concentration of 0.05 ng/mL. Cells were incubated an additional 30 minutes and then harvested. Protein was extracted and analyzed for the respective proteins shown using Western blotting.

FIG. 8. Resistance of the inhibitors to proteolytic degradation. Samples of purified inhibitors were incubated in 90% mouse serum at 37° C. Samples were removed at the indicated time points, diluted 1:10 with PBS, and analyzed by Western blotting using a polyclonal antibody raised against the rat betaglycan ectodomain (from Dr. Fernando Lopez-Casillas, UNAM, Mexico City).

### DESCRIPTION

Transforming growth factor beta (TGFβ) isoforms (β1, β2, and β3) are homodimeric polypeptides of 25 kDa. These TGFβ isoforms are secreted in a latent form and only a small percentage of total secreted TGFβs are activated under physiological conditions. TGFβ binds to three different cell surface receptors called type I (RI), type II (RII), and type III (RIII) receptors. RI and RII are serine/threonine kinase receptors. RIII (also called betaglycan) has two TGFβ binding sites in its extracellular domain, which are called the E- and U-domains, respectively. TGFβ1 and TGFβ3 bind RII with an affinity that is 200-300 fold higher than TGF-β2 (Baardsnes et al., Biochemistry, 48, 2146-55, 2009); accordingly, cells deficient in RIII are 200- to 300-fold less responsive to equivalent concentrations of TGF-β2 compared to TGF-β1 and TGFβ-3 (Chiefetz, et al (1990) J. Bio. Chem, 265, 20533-20538). However, in the presence of RIII, cells respond roughly equally to all three TGF-β isoforms, consistent with reports that show that RIII can sequester and present the ligand to RII to augment TGFβ activity when it is membrane-bound (Chen et al., J. Biol. Chem. 272, 12862-12867, 1997; Lopez-Casillas et al., Cell 73, 1435-1444, 1993; Wang et al., Cell 67, 797-805, 1991; Fukushima et al., J. Biol. Chem. 268, 22710-22715, 1993; Lopez-Casillas et al., J. Cell Biol. 124, 557-568, 1994).

Binding of TGFβ to RII recruits and activates RI through phosphorylation (Wrana et al., Nature 370, 341-347, 1994). The activated RI phosphorylates intracellular Smad2 and Smad3, which then interact with Smad4 to regulate gene expression in the nucleus (Piek et al., FASEB J. 13, 2105-2124, 1999; Massague and Chen, Genes & Development 14, 627-644, 2000). Through its regulation of gene expression, TGFβ has been shown to influence many cellular functions such as cell proliferation, cell differentiation, cell-cell and cell-matrix adhesion, cell motility, and activation of lymphocytes (Massague, Ann. Rev. Cell Biol. 6, 597-641, 1990, In Peptide growth factors and their receptors I, Sporn and Roberts, eds. (Heidelberg: Springer-Verlag), pp. 419-472, 1991). TGFβ has also been shown or implicated in inducing or mediating the progression of many diseases such as osteoporosis, hypertension, atherosclerosis, hepatic cirrhosis and fibrotic diseases of the kidney, liver, and lung (Blobe et al., N. Engl. J. Med. 342, 1350-1358, 2000). Perhaps, the most extensively studied function of TGFβ is its role in tumor progression.

TGF-β has nine cysteine residues that are conserved among its family; eight form disulfide bonds within the molecule to create a cystine knot structure characteristic of the TGF-β superfamily while the ninth cysteine forms a bond with the ninth cysteine of another TGF-β molecule to produce the dimer.

The TGF-β isoforms have been shown to promote the progression of several human diseases, such as cancer and fibrosis, yet no inhibitors have been approved for clinical use (Akhurst and Hata, 2012, Nature reviews. Drug discovery, 11, 790-811). Thus, there is an urgent need for effective and safe TGF-β inhibitors.

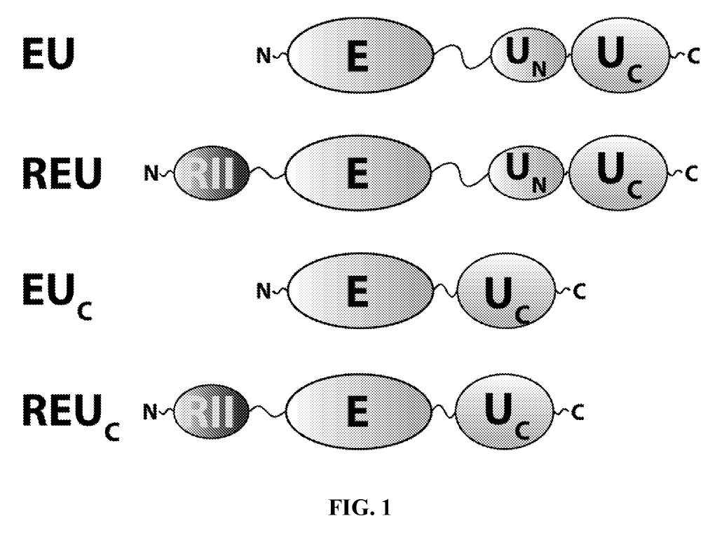

The TGF-β inhibitors described herein—REU, EU_{C}, and REU_{C}—can be produced as follows: REU is formed by artificially fusing together the binding domains of the TGF-β type II (RII or R) and type III receptor (RIII or EU) by a flexible linker, EU_{C} is formed by removing the non-binding N-terminal subdomain (U_{N}) from the uromodulin-like domain of the TGF-β type III receptor, and REU_{C} is generated by fusing together the binding domains of the TGF-β type II (RII or R) and type III receptor (RIII or EU) by a flexible linker and by removing the non-binding N-terminal subdomain (U_{N}) from the uromodulin-like domain of the TGF-β type III receptor (FIG. 1).

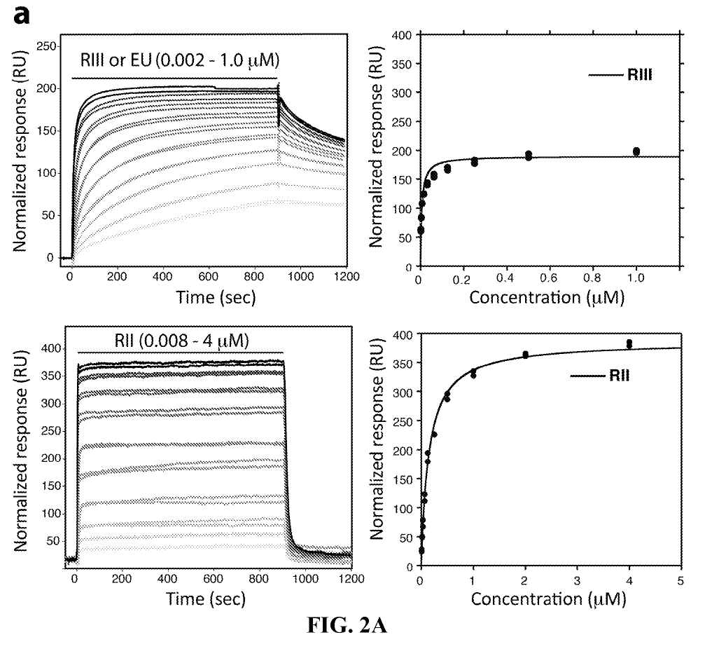

The TGF-β type III receptor binds TGF-β dimers with 1:1 stoichiometry. This was shown by comparing the maximal mass-normalized SPR response as increasing concentrations of the purified TGF-β type II receptor ectodomain (RII) and purified TGF-β type III receptor ectodomain (RIII) were injected over immobilized TGF-β2 K25R I92V K94R (TGF-β2TM), a variant of TGF-β2 that binds RII with high affinity (Baardsnes et al, 2009, Biochemistry, 48, 2146-2155; De Crescenzo et al, 2006, J Mol Biol, 355, 47-62). The maximal mass-normalized response for RIII was found to be approximately one-half of that for RII (FIG. 2a), allowing us to infer that RIII must bind the TGF-β dimer with 1:1 stoichiometry since it is well established through structural studies that RII binds TGF-β dimers with 2:1 stoichiometry (Groppe et al, 2008, Mol Cell, 29, 157-168; Hart et al, 2002, Nat Struct Biol, 9, 203-208; Radaev et al, 2010, J Biol Chem, 285, 14806-14814).

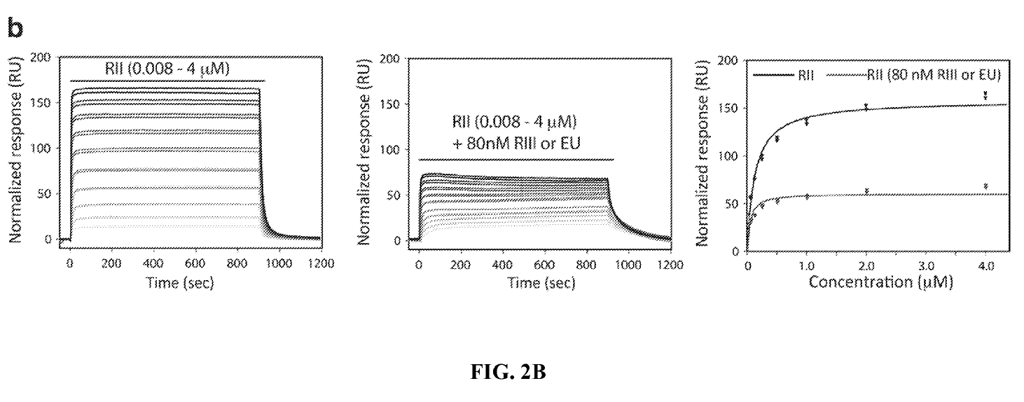

The TGF-β type III receptor potentiates the binding of the TGF-β type II receptor, but reduces its binding stoichiometry to one per TGF-β homodimer. This was shown by performing SPR experiments in which increasing concentrations of RII were injected over immobilized TGF-β2TM in the absence or presence of a saturating concentration of RIII (80 nM) (FIG. 2b). The data showed that the maximal mass normalized binding response for RII was reduced by a factor of two in the presence of 80 nM RIII (FIG. 2b), showing that one of the domains of RIII competes with RII for binding TGF-β. This indicates that TGF-βs bind one RII when RIII is bound since structural studies show that RII binds TGF-β dimers with 2:1 stoichiometry when RIII is not bound (Groppe et al, 2008, Mol Cell, 29, 157-168; Hart et al, 2002, Nat Struct Biol, 9, 203-208; Radaev et al, 2010, J Biol Chem, 285, 14806-14814). This, together with the SPR result described above, indicates that when bound together, RII, RIII, and TGF-β homodimers form a 1:1:1 complex (FIG. 3a).

RII:RIII:TGF-β2TM form a stable non-disassociating 1:1:1 complex in solution. To show that RII, RIII, and TGF-β2TM form a stable non-disassociating 1:1:1 complex in solution, 1.5 molar equivalents of 2:1 RII:TGF-β2TM complex was added to 1.0 molar equivalent of RIII (EU). The mixture was applied to a Superdex 200 size exclusion chromatography column and the UV absorbance of the column eluate at 280 nm was monitored. Three peaks were found to elute from the column, the RII:RIII:TGF-β2TM complex (peak a), excess unbound RII:TGF-β2TM complex (peak b), and excess RII (peak c) (FIG. 3b). The RII:RIII:TGF-β2TM complex isolated by size-exclusion chromatography (peak a) was found to migrate the same as the RII:RIII:TGF-β2TM complex formed from an excess of RII:TGF-β2TM complex with RIII, indicating that the isolated complex contained a full complement of the bound receptors (FIG. 3c). To determine the stoichiometry of the complex, a sample of the isolated RII:RIII:TGFβ2-TM complex was run on an SDS-PAGE gel along with known amounts of the individual components (FIG. 3d). The relative proportions of RII, RIII, and TGF-β2TM in the complex were determined by using densitometry and were found to be close to 1:1:1 (61.8, 62.4, and 51.6 pmol, respectively) (FIG. 3d).

These observations show that RII (R) and RIII (EU) form a 1:1:1 complex with TGF-β homodimers. This has led to the REU fusion as a novel inhibitor for binding and sequestering TGF-β. This fusion is a derivative of RIII in that it includes an additional N-terminal RII domain.

An example of an REU amino acid sequence (for example see SEQ ID NO:8) has the following features:

1. In certain aspects the RII sequence is human (SEQ ID NO:1), while the RIII sequence can be rat (SEQ ID NO:2).

2. In certain embodiments the N-terminal RII (R) sequence of REU extends from residue 19-136 of SEQ ID NO:1, while the C-terminal RIII (EU) sequence of REU extends from residue 31-759 of SEQ ID NO:2.

3. In certain embodiments, there is an 18 amino acid linker with the sequence Gly-Leu-Gly-Pro-Val-Glu-Ser-Ser-Pro-Gly-His-Gly-Leu-Asp-Thr-Ala-Ala-Ala (SEQ ID NO:11) that links the C-terminus of the N-terminal RII to the N-terminus of RIII.

4. In certain embodiments there is a C-terminal hexa-histidine tag (for purification purposes).

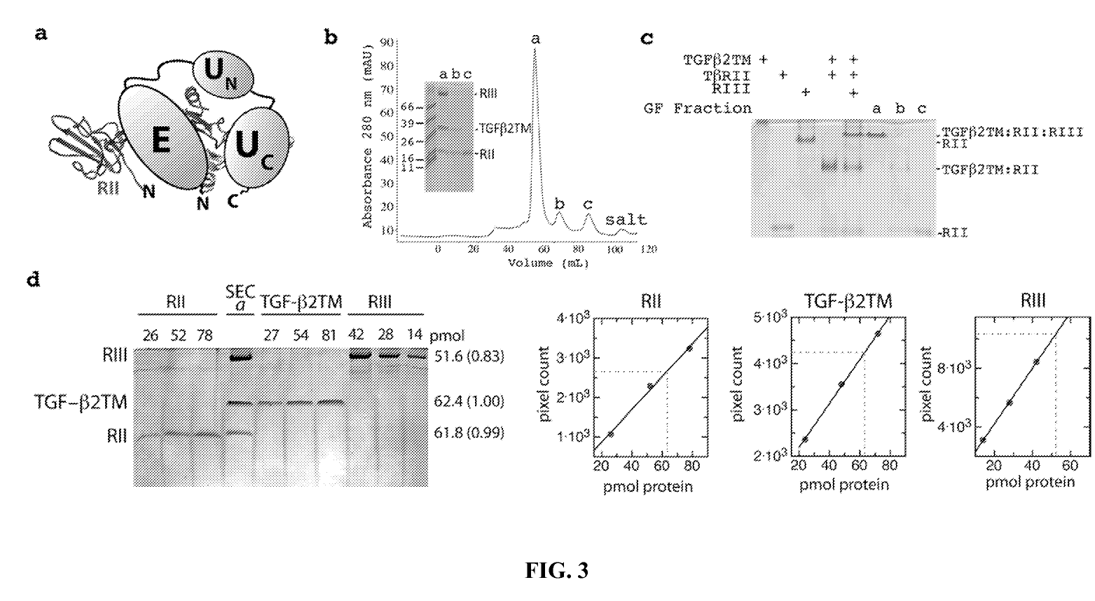

C-terminal portion of the RIII U-domain (U_{C}) binds TGF-β2TM with the same affinity as the full-length RIII U-domain (U). This was shown by performing an SPR experiment in which either the full-length RIII U-domain or just the C-terminal portion, designated U_{C}, was injected over immobilized TGF-β2TM. The concentration dependence of the response was essentially indistinguishable, indicating that all of the residues required for binding of the RIII U-domain are localized to the C-terminal subdomain, designated U_{C} (FIG. 4). This further implies that residues in the N-terminal subdomain of the U-domain, designated U_{N}, is dispensable for binding TGF-β. This led to EU_{C} and REU_{C} as novel inhibitors. These inhibitors correspond to a form of RIII (EU) and RII-RIII (REU) respectively in which the N-terminal portion of the RIII U-domain has been deleted.

An example of an EU_{C} amino acid sequence (for example see SEQ ID NO:7) has the following features:

1. In certain aspects the EU_{C} sequence is from rat (SEQ ID NO:7).

2. In certain embodiments the −terminal RIII (EU) sequence of EU_{C} sequence extends from residue 31-759, with residues 383-588 deleted of SEQ ID NO:2.

4. In certain embodiments there is a C-terminal hexa-histidine tag (for purification purposes).

An example of an REU_{C} amino acid sequence (for example see SEQ ID NO:9) has the following features:

1. In certain aspects the RII sequence is human (SEQ ID NO:1), while the RIII sequence can be rat (SEQ ID NO:2).

2. In certain embodiments the N-terminal RII sequence of REU_{C} extends from residue 19-136 of SEQ ID NO:1, while the C-terminal RIII (EU) sequence of REU extends from residue 31-759, with residues 383-588 deleted of SEQ ID NO:2.

3. In certain embodiments there is an 18 amino acid linker with the sequence Gly-Leu-Gly-Pro-Val-Glu-Ser-Ser-Pro-Gly-His-Gly-Leu-Asp-Thr-Ala-Ala-Ala (SEQ ID NO:11) that links the C-terminus of the N-terminal RII to the N-terminus of RIII.

4. In certain embodiments there is a C-terminal hexa-histidine tag (for purification purposes).

In one example, an REU, EU_{C}, REU_{C} expression cassette was inserted downstream of the albumin signal peptide and an engineered NotI cloning site with the sequence Met-Lys-Trp-Val-Thr-Phe-Leu-Leu-Leu-Leu-Phe-Ile-Ser-Gly-Ser-Ala-Phe-Ser-Ala-Ala-Ala (SEQ ID NO:10). The entire albumin signal peptide was placed downstream of the CMV promoter in a modified form of pcDNA3.1 (Invitrogen) as previously described (Zou and Sun 2004).

A plasmid expressing EU and REU construct were transfected into CHO Lec 3.2.8.1 cells (Rosenwald et at, Molecular and cellular biology, 9, 914-924) and stable transfectants were selected using MSX (Zou and Sun, 2004, Protein expression and purification, 37, 265-272). The stable transfectants were screened for high level expression of EU or REU fusion by examining the conditioned medium using a polyclonal antibody raised against the rat betaglycan ectodomain (gift from Dr. Fernando Lopez-Casillas, UNAM, Mexico City). The clone expressing EU or REU at the highest level was expanded and ultimately transferred into serum free medium for production of conditioned medium.

A plasmid expressing EU_{C} and REU_{C} construct was transiently transfected into suspension cultured HEK-293F Freestyle cells (Invitrogen, Carlsbad, Calif.) using polyethyleneimine-based transfection reagent. The cells were cultured three days post-transfection, followed by collection of the conditioned medium by centrifugation.

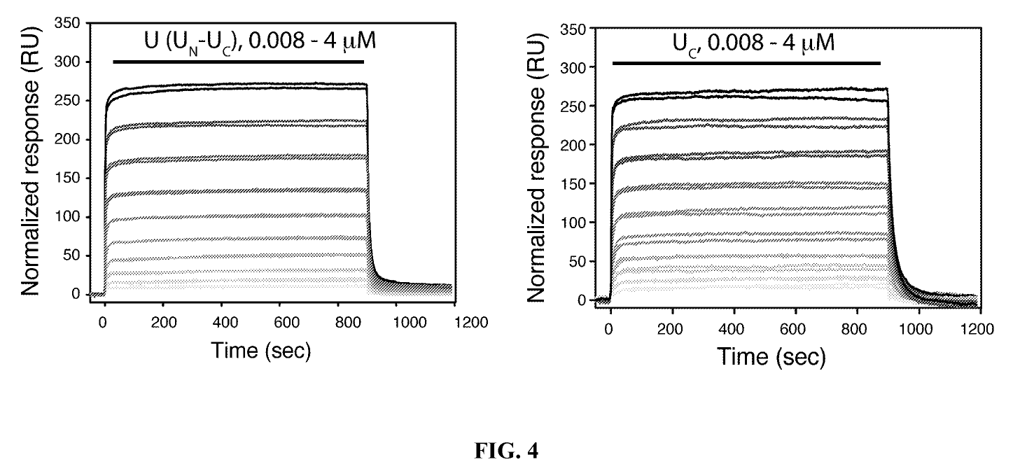

The EU, REU, REU_{C}, and EU_{C} were then purified from the conditioned medium by passing it over a NiNTA column, washing it with 25 mM Tris, 100 mM NaCl, and 7 mM imidazole, pH 8 and ultimately by eluting it with the same buffer, but with 500 mM imidazole. The nearly pure fusion proteins that eluted were concentrated and then purified to near homogeneity using size exclusion chromatography (Superdex 200, GE Healthcare) (FIG. 5).

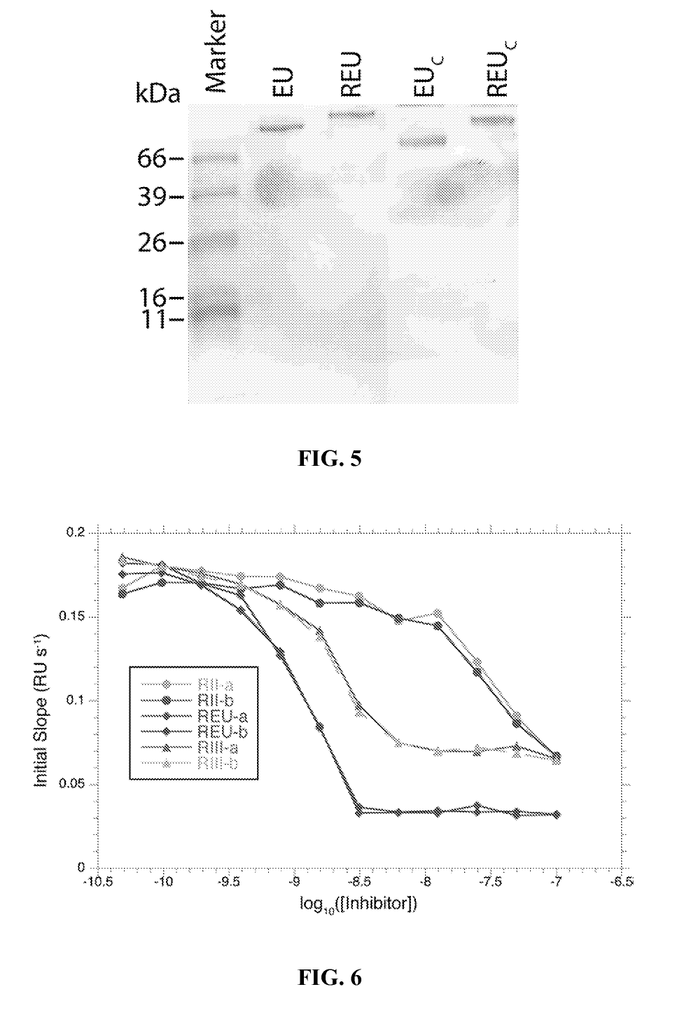

To further evaluate whether the addition of the N-terminal RII domain to RIII (EU) increased the affinity for binding TGF-β, an SPR competition experiment was performed in which the commercially available TGF-β monoclonal antibody 1D11 (R&D Systems) was coupled to an SPR sensor chip at high density (20000 RU) and in turn increasing concentration of RII, RIII (EU), or RII-RIII (REU) were injected in the presence of a fixed low (0.8 nM) concentration of TGF-β3. The initial slope of these sensorgrams, which is a linear function of the free TGF-β3 concentration, was then plotted as a function of the concentration of RII, RIII (EU), or RII-RIII (REU) (FIG. 6). This showed that REU is indeed a more potent competitor than either RII alone or RIII (EU) alone, consistent with previous finding that RII and RIII form a 1:1:1 complex with TGF-β homodimers. Though it is not possible to quantify how much more tightly REU binds TGF-β compared to RII or RIII (EU) based on this data, it is nevertheless clear that the EC₅₀ is decreased by at least 0.5 log units, or roughly 3-fold.

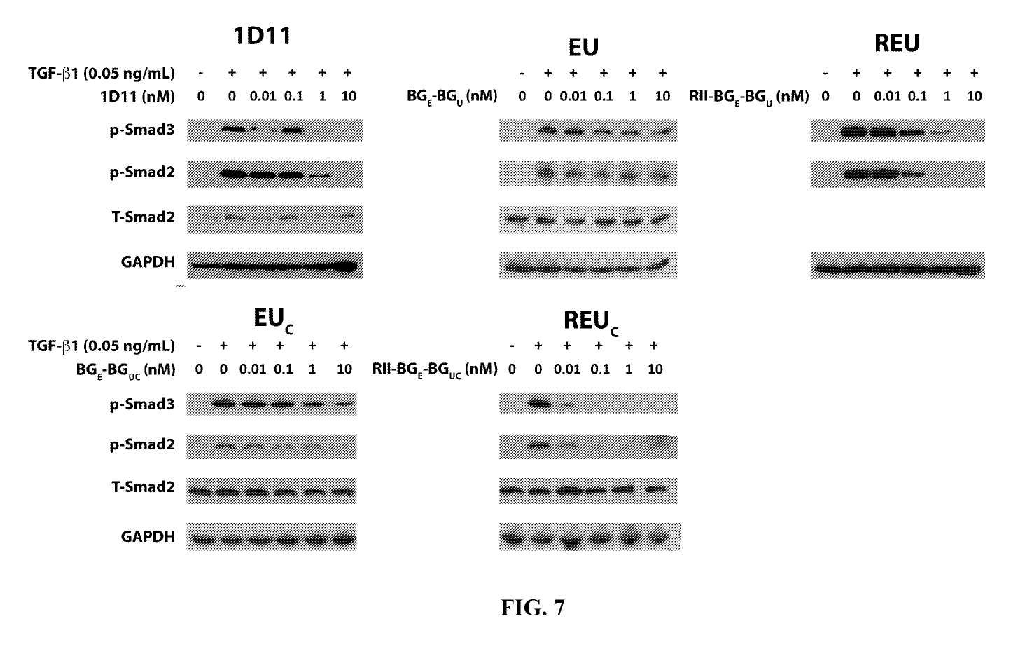

To further determine whether the increased affinity of REU for binding TGF-β translates into increased inhibitory potency (and as well whether EU_{C} and REU_{C} have increased potency compared to EU and REU), EU, EU_{C}, REU, REU_{C}, and the neutralizing antibody 1D11 have been compared in terms of their ability to antagonize TGF-β1-induced activation of Smad2 and Smad3 in cultured MDA-MB-231 human mammary epithelial cells. These measurements showed that fusion of RII onto the N-terminus of RIII and removal of the U_{N} domain individually led to an apparent increase in inhibitory potency, with REU being roughly 2-3 orders of magnitude more potent than EU and EU_{C} being 0.5-1 order of magnitude more potent than EU (FIG. 7). The fusion of RII onto the N-terminus of RIII and removal of the U_{N} domain together led to further gains in potency, with REU_{C} being roughly 2 orders of magnitude more potent than REU and roughly 4 orders of magnitude more potent than EU_{C} (FIG. 7). Together this data clearly demonstrates that each of the modifications leads to increased inhibitory potency and that the highest antagonistic potency is achieved when both modifications are introduced in concert with one another.

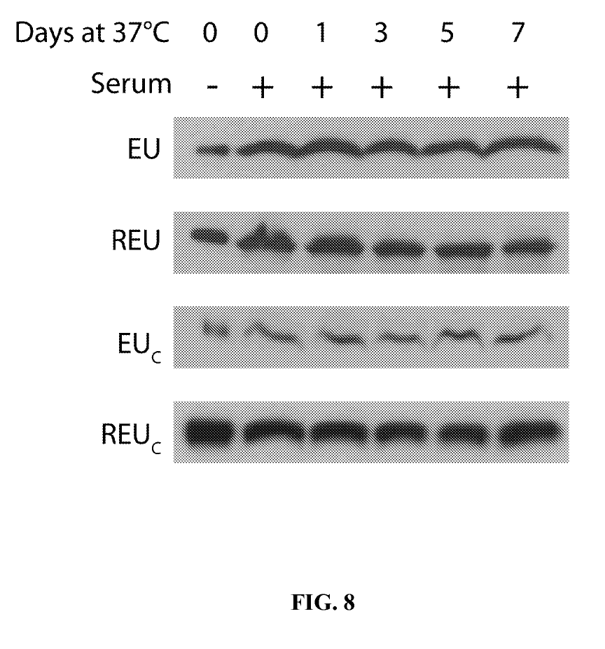

The effectiveness of these proteins in terms of attenuating the disease-promoting activity of the TGF-β isoforms in vivo will depend on their resistance to proteolytic degradation in plasma. To determine whether the inhibitors described were susceptible to proteolysis, purified samples were incubated in 90% serum obtained from Balb/c mice over a period of seven days at 37° C. The incubated samples were then diluted 1:10 in PBS and analyzed by Western blotting with a polyclonal antibody raised against the rat betaglycan ectodomain (gift from Dr. Fernando Lopez-Casillas, UNAM, Mexico City). The results showed that all of inhibitors were not detectably susceptible to proteolysis over the seven day incubation period (FIG. 8). This, along with increased potency of the inhibitors described above, is expected to contribute to the therapeutic effectiveness of these proteins in vivo.

### I. Linkers

In some embodiments, the invention provides a fusion protein comprising three TGF-β binding domains joined to each other by a linker, such as, e.g., a short peptide linker. In some embodiments, the C-terminus of the amino terminal TGF-β binding segment is joined by a short peptide linker to the N-terminus of the central TGF-β binding segment, and the C-terminus of the central TGFβ binding segment may be joined to the N-terminus of the carboxy TGFβ binding segment by a short peptide linker. A linker is considered short if it contains 2, 3, 4, 5, 6, 7, 8, 9, 10, 11, 12, 13, 14, 15, 16, 17, 18, 19, 20, 25, to 50 or fewer amino acids.

Most typically, the linker is a peptide linker that contains 50 or fewer amino acids, e.g., 45, 40, 35, 30, 25, 20, 15, 10, 9, 8, 7, 6, 5, 3, 4, 2, or 1 amino acid(s). In certain aspects, the sequence of the peptide linker is a non-TGF-β type II or type III receptor amino acid sequence. In other aspects, the sequence of the peptide linker is additional TGF-β type II or type III receptor amino acid sequence. The term additional in this context refers to amino acids in addition to those that define the segments of the heterotrimeric polypeptide as defined above. In various embodiments, the linker does not contain more than any 20, or any 10, or any 5 contiguous amino acids from the native receptor sequences. Typically, the linker will be flexible and allow the proper folding of the joined domains. Amino acids that do not have bulky side groups and charged groups are generally preferred (e.g., glycine, serine, alanine, and threonine). Optionally, the linker may additionally contain one or more adaptor amino acids, such as, for example, those produced as a result of the insertion of restriction sites. Generally, there will be no more than 10, 8, 6, 5, 4, 3, 2 adaptor amino acids in a linker.

In some embodiments, the linker comprises one or more glycines, e.g., 1, 2, 3, 4, 5, 6, 7, 8, 9, 10, 11, 12, 15, 18, or more glycines. For example, the linker may consist of (GGG)n, where n=1, 2, 3, 4, 5, 6, 7, etc. and optional adaptor amino acids. In certain aspects, the linker is a glycine-serine linker which comprises (GGGS)n, where n=1, 2, 3, 4, 5, etc. In view of the results disclosed herein, the skilled artisan will recognize that any other suitable peptide linker can be used in the fusion proteins of the invention, for example, as described in Alfthan et al., Protein Eng., 8:725-731 (1995); Argos, J. MoI. Biol., 211:943-958 (1990); Crasto et al., Protein Eng., 13:309-312 (2000); and Robinson et al., Proc. Natl. Acad. Sci. USA, 95:5929-5934 (1998).

### II. Nucleic Acids, Vectors, Host Cells

The invention further provides nucleic acids encoding any of the fusion proteins of the invention, vectors comprising such nucleic acids, and host cells comprising such nucleic acids.

Nucleic acids of the invention can be incorporated into a vector, e.g., an expression vector, using standard techniques. The expression vector may then be introduced into host cells using a variety of standard techniques such as liposome-mediated transfection, calcium phosphate precipitation, or electroporation. The host cells according to the present invention can be mammalian cells, for example, Chinese hamster ovary cells, human embryonic kidney cells (e.g., HEK 293), HeLa S3 cells, murine embryonic cells, or NSO cells. However, non-mammalian cells can also be used, including, e.g., bacteria, yeast, insect, and plant cells. Suitable host cells may also reside in vivo or be implanted in vivo, in which case the nucleic acids could be used in the context of in vivo or ex vivo gene therapy.

### III. Methods of Making

The invention also provides methods of producing (a) fusion proteins, (b) nucleic acid encoding the same, and (c) host cells and pharmaceutical compositions comprising either the fusion proteins or nucleic acids. For example, a method of producing the fusion protein according to the invention comprises culturing a host cell, containing a nucleic acid that encodes the fusion protein of the invention under conditions resulting in the expression of the fusion protein and subsequent recovery of the fusion protein. In one aspect, the fusion protein is expressed in CHO or HEK 293 cells and purified from the medium using methods known in the art. In some embodiments, the fusion protein is eluted from a column at a neutral pH or above, e.g., pH 7.5 or above, pH 8.0 or above, pH 8.5 or above, or pH 9.0 or above.

The fusion proteins, including variants, as well as nucleic acids encoding the same, can be made using any suitable method, including standard molecular biology techniques and synthetic methods, for example, as described in the following references:

Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany). Pharmaceutical compositions can also be made using any suitable method, including for example, as described in Remington: The Science and Practice of Pharmacy, eds. Gennado et al., 21th ed., Lippincott, Williams & Wilkins, 2005).

### IV. Pharmaceutical Compositions and Methods of Administration

The invention provides pharmaceutical compositions comprising the fusion proteins of the invention or nucleic acids encoding the fusion proteins.

The fusion protein may be delivered to a cell or organism by means of gene therapy, wherein a nucleic acid sequence encoding the fusion protein is inserted into an expression vector which is administered in vivo or to cells ex vivo which are then administered in vivo, and the fusion protein is expressed therefrom. Methods for gene therapy to deliver TGF-β antagonists are known (see, e.g., Fakhrai et al., Proc. Nat. Acad. Sci. USA, 93:2909-2914 (1996) and U.S. Pat. No. 5,824,655).

The fusion protein may be administered to a cell or organism in a pharmaceutical composition that comprises the fusion protein as an active ingredient. Pharmaceutical compositions can be formulated depending upon the treatment being effected and the route of administration. For example, pharmaceutical compositions of the invention can be administered orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. The pharmaceutical composition will typically comprise biologically inactive components, such as diluents, excipients, salts, buffers, preservants, etc. Standard pharmaceutical formulation techniques and excipients are well known to persons skilled in the art (see, e.g., Physicians' Desk Reference (PDR) 2005, 59th ed., Medical Economics Company, 2004; and Remington: The Science and Practice of Pharmacy, eds. Gennado et al. 21th ed., Lippincott, Williams & Wilkins, 2005).

Generally, the fusion protein of the invention may be administered as a dose of approximately from 1 μg/kg to 25 mg/kg, depending on the severity of the symptoms and the progression of the disease. The appropriate therapeutically effective dose of an antagonist is selected by a treating clinician and would range approximately from 1 μg/kg to 20 mg/kg, from 1 μg/kg to 10 mg/kg, from 1 μg/kg to 1 mg/kg, from 10 μg/kg to 1 mg/kg, from 10 μg/kg to 100 μg/kg, from 100 μg to 1 mg/kg, and from 500 μg/kg to 5 mg/kg. Effective dosages achieved in one animal may be converted for use in another animal, including human, using conversion factors known in the art (see, e.g., Freireich et al., Cancer Chemother. Reports, 50(4):219-244 (1996)).

### V. Therapeutic and Non-Therapeutic Uses

The fusion proteins of the invention may be used to capture or neutralize TGF-β, thus reducing or preventing TGF-β binding to naturally occurring TGF-β receptors.

The invention includes a method of treating a subject (e.g., mammal) by administering to the mammal a fusion protein of the invention or a nucleic acid encoding the fusion protein or cells containing a nucleic acid encoding the fusion protein. The mammal can be for example, primate (e.g., human), rodent (e.g., mouse, guinea pig, rat), or others (such as, e.g., dog, pig, rabbit).

The mammal being treated may have or may be at risk for one or more conditions associated with an excess of TGF-β for which a reduction in TGF-β levels may be desirable. Such conditions include, but are not limited to, fibrotic diseases (such as glomerulonephritis, neural scarring, dermal scarring, pulmonary fibrosis (e.g., idiopathic pulmonary fibrosis), lung fibrosis, radiation-induced fibrosis, hepatic fibrosis, myelofibrosis), peritoneal adhesions, hyperproliferative diseases (e.g., cancer), burns, immune-mediated diseases, inflammatory diseases (including rheumatoid arthritis), transplant rejection, Dupuytren's contracture, and gastric ulcers.

In certain embodiments, the fusion proteins, nucleic acids, and cells of the invention are used to treat diseases and conditions associated with the deposition of extracellular matrix (ECM). Such diseases and conditions include, but are not limited to, systemic sclerosis, postoperative adhesions, keloid and hypertrophic scarring, proliferative vitreoretinopathy, glaucoma drainage surgery, corneal injury, cataract, Peyronie's disease, adult respiratory distress syndrome, cirrhosis of the liver, post myocardial infarction scarring, restenosis (e.g., post-angioplasty restenosis), scarring after subarachnoid hemorrahage, multiple sclerosis, fibrosis after laminectomy, fibrosis after tendon and other repairs, scarring due to tatoo removal, biliary cirrhosis (including sclerosing cholangitis), pericarditis, pleurisy, tracheostomy, penetrating CNS injury, eosinophilic myalgic syndrome, vascular restenosis, veno-occlusive disease, pancreatitis and psoriatic arthropathy. In particular, the fusion proteins, and related aspects of the invention are particularly useful for the treatment of peritoneal fibrosis/adhesions. In particular, without being bound to any particular theory, animal studies in rodent models have shown poor systemic bioavailability of the fusion protein in the bloodstream following intraperitoneal administration. In contrast, it is well known that antibodies are readily transferred from the peritoneal cavity into circulation. Therefore, intraperitoneal delivery of the fusion protein may provide a highly localized form of treatment for peritoneal disorders like peritoneal fibrosis and adhesions due to the advantageous concentration of the fusion protein within the affected peritoneum as well as the associated advantage of reduced risk of complications associated with systemic delivery.

The fusion proteins, nucleic acids and cells of the invention are also useful to treat conditions where promotion of re-epithelialization is beneficial. Such conditions include, but are not limited to: diseases of the skin, such as venous ulcers, ischemic ulcers (pressure sores), diabetic ulcers, graft sites, graft donor sites, abrasions and burns; diseases of the bronchial epithelium, such as asthma and ARDS; diseases of the intestinal epithelium, such as mucositis associated with cytotoxic treatment, esophageal ulcers (reflex disease), stomach ulcers, and small intestinal and large intestinal lesions (inflammatory bowel disease).

Still further uses of the fusion proteins, nucleic acids and cells of the invention are in conditions in which endothelial cell proliferation is desirable, for example, in stabilizing atherosclerotic plaques, promoting healing of vascular anastomoses, or in conditions in which inhibition of smooth muscle cell proliferation is desirable, such as in arterial disease, restenosis and asthma.

The fusion proteins, nucleic acids and cells of the invention are also useful in the treatment of hyperproliferative diseases, such as cancers including, but not limited to, breast, prostate, ovarian, stomach, renal (e.g., renal cell carcinoma), pancreatic, colorectal, skin, lung, thyroid, cervical and bladder cancers, glioma, glioblastoma, mesothelioma, melanoma, as well as various leukemias and sarcomas, such as Kaposi's Sarcoma, and in particular are useful to treat or prevent recurrences or metastases of such tumors. In particular embodiments, the fusion proteins, nucleic acids and cells of the invention are useful in methods of inhibiting cyclosporin-mediated metastases. It will of course be appreciated that in the context of cancer therapy, “treatment” includes any medical intervention resulting in the slowing of tumor growth or reduction in tumor metastases, as well as partial remission of the cancer in order to prolong life expectancy of a patient. In one embodiment, the invention is a method of treating cancer comprising administering a fusion protein, nucleic acid or cells of the invention. In particular embodiments, the condition is renal cancer, prostate cancer or melanoma.

The fusion proteins, nucleic acids and cells of the invention are also useful for treating, preventing and reducing the risk of occurrence of renal insufficiencies including, but not limited to, diabetic (type I and type II) nephropathy, radiational nephropathy, obstructive nephropathy, diffuse systemic sclerosis, pulmonary fibrosis, allograft rejection, hereditary renal disease (e.g., polycystic kidney disease, medullary sponge kidney, horseshoe kidney), nephritis, glomerulonephritis, nephrosclerosis, nephrocalcinosis, systemic lupus erythematosus, Sjogren's syndrome, Berger's disease, systemic or glomerular hypertension, tubulointerstitial nephropathy, renal tubular acidosis, renal tuberculosis, and renal infarction. In particular embodiments, the fusion proteins, nucleic acids and cells of the invention are combined with antagonists of the renin-angiotensin-aldosterone system including, but not limited to, renin inhibitors, angiotensin-converting enzyme (ACE) inhibitors, Ang Ii receptor antagonists (also known as “Ang Il receptor blockers”), and aldosterone antagonists (see, for example, WO 2004/098637).

The fusion proteins, nucleic acids and cells of the invention are also useful to enhance the immune response to macrophage-mediated infections, such as those caused by Leishmania spp., Trypanosoma cruzi, Mycobacterium tuberculosis and Mycobacterium leprae, as well as the protozoan Toxoplasma gondii, the fungi Histoplasma capsulatum, Candida albicans, Candida parapsilosis, and Cryptococcus neoformans, and Rickettsia, for example, R. prowazekii, R. coronii, and R. tsutsugamushi. They are also useful to reduce immunosuppression caused, for example, by tumors, AIDS or granulomatous diseases.

In certain embodiments, the fusion proteins, nucleic acids and cells of the invention are used to treat diseases and conditions in which a TGF-β antagonist that is smaller in size and/or has a shorter half-life, relative to other TGF-β antagonists, is more effective as a therapeutic agent. As described herein, the fusion proteins of the invention are smaller than other TGF-β antagonists (e.g., TGF-β antibodies, TGF-β receptor-Fc fusion proteins) and have a shorter circulatory half-life. Accordingly, such fusion proteins may show increased efficacy in treating diseases or conditions where such characteristics are desirable. For example, without being bound to any particular theory, it is believed that the fusion proteins of the invention, because of their small size relative to other TGF-G antagonists, may exhibit increased targeting to sites of action (e.g., increased penetration of tumors, increased penetration of tissue (e.g., fibrotic tissue)).

In addition, without being bound to any particular theory, it is also believed that the fusion proteins of the invention, because they lack an immunoglobulin domain (unlike TGF-β antibodies and TGF-β receptor-Fc fusion proteins) may not be as susceptible to clearance from sites of action by the immune system (e.g., in conditions or diseases of the lung).

As described herein and is known in the art, TGF-β is involved in many cellular processes including cell growth, cell differentiation, apoptosis, cellular homeostasis and other cellular functions. Given the crucial role that TGF-β has in cellular processes, there may be conditions or diseases in which it is preferable to administer a shorter-acting TGF-β antagonist, which correspondingly would have fewer negative associated effects than a longer-acting TGF-β antagonist (such as a TGF-β antibody or TGF-β receptor-Fc fusion protein). Accordingly, without being bound to any particular theory, it is believed that the fusion proteins of the invention, because of their shorter circulating half-life, may exhibit fewer negative TGF-β antagonist-related effects.

1. A heteromeric fusion protein comprising from amino terminus to carboxy terminus:
(a) an amino terminal ectodomain of TGFβ receptor type II domain, a TGFβ receptor type III endoglin domain, and a TGFβ receptor type III uromodulin-like carboxy terminal binding subdomain (U_{C}); or
(b) an amino terminal TGFβ receptor type III endoglin domain coupled to a TGFβ receptor type III uromodulin-like carboxy terminal binding subdomain (U_{C}).

2. The fusion protein of claim 1, further comprising one or more linker amino acids between (i) the amino terminal ectodomain of TGFβ receptor type II and the TGFβ receptor type III endoglin domain, (ii) the TGFβ receptor type III endoglin domain and the uromodulin-like carboxy-terminal binding subdomain, or (iii) both the amino terminal ectodomain of TGFβ receptor type II and the TGFβ receptor type III endoglin domain, and the TGFβ receptor type III endoglin domain and the uromodulin-like carboxy-terminal binding subdomain

3. The fusion protein of claim 1, further comprising an amino terminal signal sequence.

4. The fusion protein of claim 1, further comprising an amino terminal or carboxy terminal tag.

5. The fusion protein of claim 1, wherein the tag is a carboxy terminal hexa-histidine

6. A method of treating a condition related to increased expression TGFβ comprising administering an effective amount of the fusion protein of claim 1 to subject in need thereof.

7. The method of claim 6, wherein the condition is a hyperproliferative disorder.

8. The method of claim 7, wherein the hyperproliferative disorder is cancer.
